(19) United States
(12) Patent Application Publication
(10) Pub. No.: US 2015/0135372 A1
(43) Pub. Date: May 14, 2015

(54) Transgenic Plants With Enhanced Agronomic Traits

(71) Applicant: MONSANTO TECHNOLOGY LLC, St. Louis, MO (US)

(72) Inventors: Edwards M Allen, O'Fallon, MO (US); Sergey Ivashuta, Ballwin, MO (US); Molian Deng, Glencoe, MO (US); Brian M Hauge, Wildwood, MO (US); Huai Wang, Chesterfield, MO (US)

(73) Assignee: MONSANTO TECHNOLOGY LLC, St. Louis, MO (US)

(21) Appl. No.: 14/610,728

(22) Filed: Jan. 30, 2015

Related U.S. Application Data
(63) Continuation of application No. 13/926,621, filed on Jun. 25, 2013.
Application No. 13/926,621 is a continuation of application No. 13/122,581, filed on Jun. 13, 2011, now abandoned.
(60) Provisional application No. 61/103,594, filed on Oct. 8, 2008.

(51) Int. Cl. C12N 15/82

(52) U.S. Cl. CPC C12N 15/8218; C12N 15/8274; C12N 15/8275; C12N 15/8261
USPC 800/300.1; 435/320.1; 800/298; 800/320.2; 800/320.3; 800/320.1; 435/419; 435/418; 800/300

(57) ABSTRACT

This invention provides transgenic plant cells with recombinant DNA for expression of proteins that are useful for imparting enhanced agronomic trait(s) to transgenic crop plants. This invention also provides transgenic plants and progeny seed comprising the transgenic plant cells where the plants are selected for having an enhanced trait selected from the group of traits consisting of enhanced water use efficiency, enhanced cold tolerance, increased yield, enhanced nitrogen use efficiency, enhanced seed protein and enhanced seed oil. Also disclosed are methods for manufacturing transgenic seed and plants with enhanced traits.

### CROSS REFERENCE TO RELATED APPLICATIONS

This application claims benefit under 35 USC §119(e) of U.S. provisional application Ser. No. 61/103,594, filed Oct. 8, 2008, which is incorporated herein by reference in its entirety.

### INCORPORATION OF SEQUENCE LISTING

The sequence listing that is contained in the file named “38_{—}21_{—}56300_B_seqListing.txt”, which is 3,450,299 bytes (measured in MS-WINDOWS), created on Oct. 3, 2009 is filed herewith by electronic submission and incorporated herein by reference.

### FIELD OF THE INVENTION

Disclosed herein are recombinant DNA useful for providing enhanced traits to transgenic plants, seeds, pollen, plant cells and plant nuclei of such transgenic plants, methods of making and using such recombinant DNA, plants, seeds, pollen, plant cells and plant nuclei. Also disclosed are methods of producing hybrid corn seed comprising such recombinant DNA.

### SUMMARY OF THE INVENTION

In some aspects, this invention provides recombinant DNA constructs which comprise polynucleotides which modulate the expression of a protein without producing offtypes resulting from complete loss of the protein's function (e.g. through homozygous recessive mutations in the genomic DNA encoding the protein). This invention also provides recombinant DNA constructs comprising a polynucleotide which suppresses a protein having an SBP pfam domain scoring above 25.000. In specific embodiments, this invention provides a recombinant DNA construct comprising a polynucleotide which suppresses a protein having 95% identity over 95% of the length of SEQ ID NO: 19 where the recombinant DNA construct comprises a polynucleotide which encodes a miRNA. This invention also provides recombinant DNA constructs comprising a polynucleotide which encodes a protein that has an amino acid sequence having at least 95% identity over at least 95% of the length of a reference sequence selected from the group of sequences consisting of SEQ ID NO: 16-21 when the amino acid sequence is aligned with the reference sequence, or which is transcribed into a precursor of a miRNA that has the function of a miRNA with a sequence selected from SEQ ID NOs: 1-2, 7, 9-13, and 15. The recombinant DNA constructs are useful for providing enhanced traits when stably integrated into the chromosomes and expressed in transgenic plants cells. In further aspects, the invention provides plants cells comprising stably integrated recombinant DNA constructs of the invention. In particularly useful aspects of the invention, plant cells comprise stably integrated recombinant DNA constructs which include polynucleotides operably linked to a promoter which is functional in the plant cells. In some aspects of the invention, expression of the polynucleotides in the plant cells effect modulation of the expression of a protein. In other aspects of the invention, expression of the polynucleotides in the plant cells effect modulation of the activity of a miRNA. In some aspects, expression of the polynucleotides in the recombinant DNA of the invention is used to enhance expression of a protein, i.e. a protein that has at least 95% identity over at least 95% of the length of a reference sequence selected from SEQ ID NOs: 18 and 20 or to suppress expression of a protein, i.e. a protein having the function of a protein with a sequence selected from SEQ ID NOs: 16-17, 19, and 21. Likewise, in other aspects, expression of the polynucleotides in the recombinant DNA of the invention is used to enhance the activity of a miRNA having the function of a miRNA with a sequence selected from SEQ ID NOs: 1, 2, and 7, or to suppress the activity of a miRNA with the function of a miRNA having a sequence selected from SEQ ID NOs: 9-13 and 15. Polynucleotides in aspects of the invention used for enhancement of protein expression includes DNA which is transcribed into: (a) messenger RNA encoding the protein, (b) a miRNA decoy for a miRNA which targets a messenger RNA encoding the protein, (c) a messenger RNA which encodes the protein and is resistant to miRNA-mediated suppression, and (d) a small RNA which prevents cleavage of a messenger RNA encoding the protein. Polynucleotides in aspects of the invention used for suppression of protein expression includes DNA which is transcribed into: (a) a dsRNA which is processed into siRNAs which target a messenger RNA encoding the protein, (b) a miRNA that targets a messenger RNA encoding the protein, (c) a messenger RNA which encodes the protein and is sensitive to miRNA-mediated suppression, and (d) a trans-acting (ta) siRNA which is processed into siRNAs which target a messenger RNA encoding the protein. Polynucleotides in aspects of the invention used for enhancement of miRNA activity, includes DNA which is transcribed into RNA which encodes a miRNA. Polynucleotides in aspects of the invention used for suppression of miRNA activity includes DNA which is transcribed into a miRNA decoy for the miRNA.

This invention also provides transgenic plants comprising a plurality of transgenic plant cells of the invention, and transgenic seeds and transgenic pollen of such plants. Such transgenic plants are selected from a population of transgenic plants regenerated from plant cells transformed with recombinant DNA by screening transgenic plants for an enhanced trait as compared to control plants. The enhanced trait is one or more of enhanced water use efficiency, enhanced cold tolerance, increased yield, enhanced nitrogen use efficiency, enhanced seed protein and enhanced seed oil.

In another aspect of the invention the plant cells, plants, seeds, and pollen further comprise DNA expressing a protein that provides tolerance from exposure to an herbicide applied at levels that are lethal to a wild type plant cell.

This invention also provides methods for manufacturing non-natural, transgenic seed that can be used to produce a crop of transgenic plants with an enhanced trait resulting from expression of stably-integrated, recombinant DNA. More specifically, the method comprises (a) screening a population of plants for an enhanced trait and a recombinant DNA, where individual plants in the population can exhibit the trait at a level less than, essentially the same as or greater than the level that the trait is exhibited in control plants, (b) selecting from the population one or more plants that exhibit the trait at a level greater than the level that said trait is exhibited in control plants, (c) collecting seed from a selected plant, (d) verifying that the recombinant DNA is stably integrated in said selected plants, (e) analyzing tissue of a selected plant to determine the production or suppression of a protein having the function of a protein encoded by nucleotides in a sequence of one of SEQ ID NOs:16-21 or a miRNA having the function of a miRNA encoded by the nucleotides in a sequence of one of SEQ ID NOs: 1,2,7, 9-13, and 15. In one aspect of the invention, the plants in the population further comprise DNA expressing a protein that provides tolerance to exposure to a herbicide applied at levels that are lethal to wild type plant cells and the selecting is affected by treating the population with the herbicide, e.g. a glyphosate, dicamba, or glufosinate compound. In another aspect of the invention the plants are selected by identifying plants with the enhanced trait. The methods are especially useful for manufacturing corn, soybean, cotton, canola, alfalfa, wheat, rice, sugarcane or sugar beet seed.

Another aspect of the invention provides a method of producing hybrid corn seed comprising acquiring hybrid corn seed from a herbicide tolerant corn plant which also has stably-integrated, recombinant DNA comprising a promoter that is (a) functional in plant cells and (b) is operably linked to DNA that enhances or suppresses the expression of a protein having the function of a protein encoded by nucleotides in a sequence of one of SEQ ID NOs: 16-21 or which enhances or suppresses the activity of a miRNA having the function of a miRNA encoded by nucleotides in a sequence of one of SEQ ID NOs: 1,2,7, 9-13, and 15. The methods further comprise producing corn plants from said hybrid corn seed, wherein a fraction of the plants produced from said hybrid corn seed is homozygous for said recombinant DNA, a fraction of the plants produced from said hybrid corn seed is hemizygous for said recombinant DNA, and a fraction of the plants produced from said hybrid corn seed has none of said recombinant DNA; selecting corn plants which are homozygous and hemizygous for said recombinant DNA by treating with an herbicide; collecting seed from herbicide-treated-surviving corn plants and planting said seed to produce further progeny corn plants; repeating the selecting and collecting steps at least once to produce an inbred corn line; and crossing the inbred corn line with a second corn line to produce hybrid seed.

Another aspect of the invention provides a method of selecting a plant comprising plant cells of the invention by using an immunoreactive antibody to detect the presence or absence of protein expressed or suppressed by recombinant DNA in seed or plant tissue. Yet another aspect of the invention provides anti-counterfeit milled seed having, as an indication of origin, plant cells of this invention.

In unique aspects of the invention, the transgenic plants exhibit enhanced traits such as enhanced yield, enhanced yield under stress e.g. water deficit stress, nitrogen deficit stress, and cold stress as compared to control plants.

### BRIEF DESCRIPTION OF THE DRAWINGS

FIG. 1 shows a comparison of the leaf angle of corn plants with and without a synthetic miRNA for the suppression of LG1 expression.

### DETAILED DESCRIPTION OF THE INVENTION

In the attached sequence listing:

SEQ ID NO: 1-2, 7, 9-13, and 15 are nucleotide sequences of miRNAs;

SEQ ID NO:3-6, 8, and 14 are nucleotide sequences of the coding strand of DNA for “genes” used in the recombinant DNA imparting an enhanced trait in plant cells, i.e. each represents a coding sequence for a protein;

SEQ ID NO: 16-21 are amino acid sequences of the cognate protein of the “genes” with nucleotide coding sequences 3-6, 8, and 14;

SEQ ID NO: 22 is a nucleotide sequence of a base plasmid vector useful for corn transformation;

SEQ ID NO: 23 is a nucleotide sequence of a base plasmid vector useful for soybean and canola transformation;

SEQ ID NO: 24 is a nucleotide sequence of a base plasmid vector useful for cotton transformation;

SEQ ID NOs: 25-894 are sequences of proteins homologous to SEQ ID NOs: 16-21;

SEQ ID NOs: 895-1126 are nucleotide sequences of miRNAs homologous to SEQ ID NOs: 1-2, 7, 9-13, and 15.

SEQ ID NO: 1127 is a consensus sequence of SEQ ID NO: 16 and its homologs.

SEQ ID NOs: 1128-1140 are sequences of polynucleotides used to modulate expression of proteins or miRNAs of the invention.

As used herein a “plant cell” means a plant cell that is transformed with stably-integrated, non-natural, recombinant DNA, e.g. by Agrobacterium-mediated transformation or by bombardment using microparticles coated with recombinant DNA or other means. A plant cell of this invention can be an originally-transformed plant cell that exists as a microorganism or as a progeny plant cell that is regenerated into differentiated tissue, e.g. into a transgenic plant with stably-integrated, non-natural recombinant DNA, or seed or pollen derived from a progeny transgenic plant.

As used herein a “transgenic plant” means a plant whose genome has been altered by the stable integration of recombinant DNA. A transgenic plant includes a plant regenerated from an originally-transformed plant cell and progeny transgenic plants from later generations or crosses of a transformed plant.

As used herein “recombinant DNA” means DNA which has been a genetically engineered and constructed outside of a cell.

As used herein “consensus sequence” means an artificial sequence of amino acids in a conserved region of an alignment of amino acid sequences of homologous proteins, e.g. as determined by a CLUSTALW alignment of amino acid sequence of homolog proteins.

As used herein a “homolog” means a protein or miRNA in a group of proteins or miRNAs that perform the same biological function, e.g. proteins that belong to the same Pfam protein family and that provide a common enhanced trait in transgenic plants of this invention. Homologs are expressed by homologous genes. With reference to homologous genes, homologs include orthologs, i.e. genes expressed in different species that evolved from a common ancestral genes by speciation and encode proteins retain the same function, but do not include paralogs, i.e. genes that are related by duplication but have evolved to encode proteins with different functions. Homologous genes include naturally occurring alleles and artificially-created variants. Degeneracy of the genetic code provides the possibility to substitute at least one base of the protein encoding sequence of a gene with a different base without causing the amino acid sequence of the polypeptide produced from the gene to be changed. When optimally aligned, homolog proteins have at least 60%, 65%, 70%, 75%, 80%, 85%, 90%, 95%, 96%, 97%, 98%, or 99% identity over the full length of a protein identified as being associated with imparting an enhanced trait when expressed in plant cells. In one aspect of the invention homolog proteins have an amino acid sequence that has at least 90% identity to a consensus amino acid sequence of proteins and homologs disclosed herein. Genes for homologous miRNAs include any gene whose expression produces a miRNA having the function of a miRNA disclosed in table 1 such as the homologous miRNAs disclosed in table 7.

Homologous proteins are identified by comparison of amino acid sequence, e.g. manually or by use of a computer-based tool using known homology-based search algorithms such as those commonly known and referred to as BLAST, FASTA, and Smith-Waterman. A local sequence alignment program, e.g. BLAST, can be used to search a database of sequences to find similar sequences, and the summary Expectation value (E-value) used to measure the sequence base similarity. Because a protein hit with the best E-value for a particular organism may not necessarily be an ortholog, i.e. have the same function, or be the only ortholog, a reciprocal query is used to filter hit sequences with significant E-values for ortholog identification. The reciprocal query entails search of the significant hits against a database of amino acid sequences from the base organism that are similar to the sequence of the query protein. A hit can be identified as an ortholog, when the reciprocal query's best hit is the query protein itself or a protein encoded by a duplicated gene after speciation. A further aspect of the homologs encoded by DNA useful in the transgenic plants of the invention are those proteins that differ from a disclosed protein as the result of deletion or insertion of one or more amino acids in a native sequence.

Homologous miRNAs are identified by sequence comparison coupled to a comparison of secondary structure. Sequence databases are searched for regions having few (e.g. 1, 2, or 3) mismatches to a known miRNA. These regions are then examined for the potential to form a fold-back structure producing a similar pattern of matched and mismatched bases (e.g. using RNAfold from EMBOSS) to that found in the fold-back structure of the pre-miRNA for the known miRNA. Regions having few mismatches in the mature miRNA sequences and forming a similar fold-back structure are identified as homologous miRNAs.

“Percent identity” describes the extent to which the sequences of DNA or protein segments are invariant throughout a window of alignment of sequences, for example nucleotide sequences or amino acid sequences. An “identity fraction” for a sequence aligned with a reference sequence is the number of identical components which are shared by the sequences, divided by the length of the alignment not including gaps introduced by the alignment algorithm. “Percent identity” (“% identity”) is the identity fraction times 100. Percent identity is calculated over the aligned length preferably using a local alignment algorithm, such as BLASTp. As used herein, sequences are “aligned” when the alignment produced by BLASTp has a minimal e-value.

“Pfam” is a large collection of multiple sequence alignments and hidden Markov models covering many common protein families, e.g. Pfam version 19.0 (December 2005) contains alignments and models for 8183 protein families and is based on the Swissprot 47.0 and SP-TrEMBL 30.0 protein sequence databases. See S. R. Eddy, “Profile Hidden Markov Models”, Bioinformatics 14:755-763, 1998. The Pfam database is currently maintained and updated by the Pfam Consortium. The alignments represent some evolutionary conserved structure that has implications for the protein's function. Profile hidden Markov models (profile HMMs) built from the protein family alignments are useful for automatically recognizing that a new protein belongs to an existing protein family even if the homology by alignment appears to be low.

Protein domains are identified by querying the amino acid sequence of a protein against Hidden Markov Models which characterize protein family domains (“Pfam domains”) using HMMER software, which is available from the Pfam Consortium. The HMMER software is also disclosed in patent application publication US 2008/0148432 A1 incorporated herein by reference. A protein domain meeting the gathering cutoff for the alignment of a particular Pfam domain is considered to contain the Pfam domain.

A “Pfam domain module” is a representation of Pfam domains in a protein, in order from N terminus to C terminus. In a Pfam domain module individual Pfam domains are separated by double colons “::”. The order and copy number of the Pfam domains from N to C terminus are attributes of a Pfam domain module. Although the copy number of repetitive domains is important, varying copy number often enables a similar function. Thus, a Pfam domain module with multiple copies of a domain should define an equivalent Pfam domain module with variance in the number of multiple copies. A Pfam domain module is not specific for distance between adjacent domains, but contemplates natural distances and variations in distance that provide equivalent function. The Pfam database contains both narrowly- and broadly-defined domains, leading to identification of overlapping domains on some proteins. A Pfam domain module is characterized by non-overlapping domains. Where there is overlap, the domain having a function that is more closely associated with the function of the protein (based on the E value of the Pfam match) is selected.

Once one DNA is identified as encoding a protein which imparts an enhanced trait when expressed in transgenic plants, other DNA encoding proteins with the same Pfam domain module are identified by querying the amino acid sequence of protein encoded by candidate DNA against the Hidden Markov Models which characterizes the Pfam domains using HMMER software. Candidate proteins meeting the same Pfam domain module are in the protein family and have cognate DNA that is useful in constructing recombinant DNA for the use in the plant cells of this invention. Hidden Markov Model databases for use with HMMER software in identifying DNA expressing protein with a common Pfam domain module for recombinant DNA in the plant cells of this invention are available from the Pfam Consortium (ftp.sanger.ac.uk/pub/databases/Pfam/) and are incorporated herein by reference.

The HMMER software and Pfam databases (version 23.0) were used to identify known domains in the proteins corresponding to amino acid sequence of SEQ ID NOs: 17-21. All DNA encoding proteins that have scores higher than the gathering cutoff disclosed in Table 11 by Pfam analysis disclosed herein can be used in recombinant DNA of the plant cells of this invention, e.g. for selecting transgenic plants having enhanced agronomic traits. The relevant Pfams modules for use in this invention, as more specifically disclosed below, are Homeobox::HALZ, SRF-TF::K-box, SBP, and HMGL-like::LeuA_dimer for which databases are available from the Pfam Consortium (ftp.sanger.ac.uk/pub/databases/Pfam/) and are incorporated herein by reference.

As used herein “promoter” means regulatory DNA for initializing transcription. A “plant promoter” is a promoter capable of initiating transcription in plant cells whether or not its origin is a plant cell, e.g. is it well known that Agrobacterium promoters are functional in plant cells. Thus, plant promoters include promoter DNA obtained from plants, plant viruses and bacteria such as Agrobacterium and Bradyrhizobium bacteria. Examples of promoters under developmental control include promoters that preferentially initiate transcription in certain tissues, such as leaves, roots, or seeds. Such promoters are referred to as “tissue preferred”. Promoters that initiate transcription only in certain tissues are referred to as “tissue specific”. A “cell type” specific promoter primarily drives expression in certain cell types in one or more organs, for example, vascular cells in roots or leaves. An “inducible” or “repressible” promoter is a promoter which is under environmental control. Examples of environmental conditions that may effect transcription by inducible promoters include anaerobic conditions, or certain chemicals, or the presence of light. Tissue specific, tissue preferred, cell type specific, and inducible promoters constitute the class of “non-constitutive”promoters. A “constitutive” promoter is a promoter which is active under most conditions.

As used herein “operably linked” means the association of two or more DNA fragments in a recombinant DNA construct so that the function of one, e.g. protein-encoding DNA, is controlled by the other, e.g. a promoter.

As used herein “expressed” means produced, e.g. a protein is expressed in a plant cell when its cognate DNA is transcribed to mRNA that is translated to the protein.

As used herein “suppressed” means decreased, e.g. a protein is suppressed in a plant cell when there is a decrease in the amount and/or activity of the protein in the plant cell. The presence or activity of the protein can be decreased by any amount up to and including a total loss of protein expression and/or activity.

As used herein a “control plant” means a plant that does not contain the recombinant DNA that expressed a protein that impart an enhanced trait. A control plant is to identify and select a transgenic plant that has an enhance trait. A suitable control plant can be a non-transgenic plant of the parental line used to generate a transgenic plant, i.e. devoid of recombinant DNA. A suitable control plant may in some cases be a progeny of a hemizygous transgenic plant line that is does not contain the recombinant DNA, known as a negative segregant.

As used herein an “enhanced trait” means a characteristic of a transgenic plant that includes, but is not limited to, an enhance agronomic trait characterized by enhanced plant morphology, physiology, growth and development, yield, nutritional enhancement, disease or pest resistance, or environmental or chemical tolerance. In more specific aspects of this invention enhanced trait is selected from group of enhanced traits consisting of enhanced water use efficiency, enhanced cold tolerance, increased yield, enhanced nitrogen use efficiency, enhanced seed protein and enhanced seed oil. In an important aspect of the invention the enhanced trait is enhanced yield including increased yield under non-stress conditions and increased yield under environmental stress conditions. Stress conditions may include, for example, drought, shade, fungal disease, viral disease, bacterial disease, insect infestation, nematode infestation, cold temperature exposure, heat exposure, osmotic stress, reduced nitrogen nutrient availability, reduced phosphorus nutrient availability and high plant density. “Yield” can be affected by many properties including without limitation, plant height, pod number, pod position on the plant, number of internodes, incidence of pod shatter, grain size, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Yield can also be affected by efficiency of germination (including germination in stressed conditions), growth rate (including growth rate in stressed conditions), ear number, seed number per ear, seed size, composition of seed (starch, oil, protein) and characteristics of seed fill.

Increased yield of a transgenic plant of the present invention can be measured in a number of ways, including test weight, seed number per plant, seed weight, seed number per unit area (i.e. seeds, or weight of seeds, per acre), bushels per acre, tons per acre, or kilo per hectare. For example, corn yield may be measured as production of shelled corn kernels per unit of production area, for example in bushels per acre or metric tons per hectare, often reported on a moisture adjusted basis, for example at 15.5 percent moisture. Increased yield may result from improved utilization of key biochemical compounds, such as nitrogen, phosphorous and carbohydrate, or from improved responses to environmental stresses, such as cold, heat, drought, salt, and attack by pests or pathogens. Recombinant DNA used in this invention can also be used to provide plants having improved growth and development, and ultimately increased yield, as the result of modified expression of plant growth regulators or modification of cell cycle or photosynthesis pathways. Also of interest is the generation of transgenic plants that demonstrate enhanced yield with respect to a seed component that may or may not correspond to an increase in overall plant yield; such properties include enhancements in seed oil, seed molecules such as protein and starch, oil components as may be manifest by an alterations in the ratios of seed components.

The modulation of protein in transgenic plant cells (hereafter generally referred to as the “target protein”) can be achieved by a variety of approaches involving the use of recombinant DNA constructs. Examples of such recombinant DNA constructs include recombinant DNA constructs that produce messenger RNA for the target protein where native miRNA recognition sites in the mRNA for the target protein are modified or deleted, recombinant DNA constructs that produce an RNA gene suppression element such as a miRNA or a dsRNA comprising sense and anti-sense sequences from the gene encoding the target protein, recombinant DNA constructs that produce a transacting short interfering RNA (ta-siRNA) and recombinant DNA constructs that produce a miRNA element such as a decoy miRNA that is a target for native miRNA or RNA that sequesters target messenger RNA away from native miRNA.

Small RNAs that regulate protein expression include miRNAs and ta-siRNAs. A miRNA is a small (typically about 21 nucleotide) RNA that has the ability to modulate the expression of a target gene by binding to messenger RNA for the target protein leading to destabilization of the target protein messenger RNA or translational inhibition of the target protein messenger RNA, ultimately resulting in reduction of the target protein. The design and construction of ta-siRNA constructs and their use in the modulation of protein in transgenic plant cells is disclosed by Allen and Carrington in US Patent Application Publication US 2006/0174380 A1 which is incorporated herein by reference. The expression or suppression of such small RNAs are aspects of the invention that are conveniently illustrated by reference to use of miRNAs.

Recombinant DNA constructs can be used to modify the activity of native miRNAs by a variety of means. By increasing the expression of a miRNA, e.g. temporally or spatially, the modulation of expression of a native target gene can be enhanced. An alternative gene suppression approach for suppressing the expression of a target protein can include the use of a recombinant DNA construct that produces a synthetic miRNA that is designed to bind to a native or synthetic miRNA recognition site on messenger RNA for the target protein.

By reducing the expression of a miRNA, the modulation of a native target gene can be diminished resulting in enhanced expression of the target protein. More specifically, the expression of a target protein can be enhanced by suppression of the activity of the miRNA that binds to a recognition site in the messenger RNA that is transcribed from the native gene for the target protein. Several types of recombinant DNA constructs can be designed to suppress the activity of a miRNA.

For example, a recombinant DNA construct that produces an abundance of RNA with the miRNA recognition site can be used as a decoy for the native miRNA allowing endogenous messenger RNA with the miRNA recognition site to be translated to the target protein without interference from native miRNA. A recombinant DNA construct that produces RNA with a modified miRNA recognition site, e.g. with nucleotides at positions 10 and/or 11 in a 21mer miRNA recognition site which are unpaired with respect to the native miRNA, can be used to sequester natively expressed miRNA thereby reducing the cleavage that normally occurs when miRNA binds to a recognition site. The unpaired nucleotides can be produced e.g. through additional nucleotides between positions 10 and 11 or through substitutions of the nucleotides at positions 10 and 11.

Additionally, a recombinant DNA construct can be created that produces RNA that can be processed in plants into synthetic small RNA (miRNA-like) that can bind endogenous miRNA recognition sites but is unable to induce cleavage of mRNA because the small RNA is modified, for instance by having a modified nucleotide at positions 10 and/or 11 or a deletion that produces a bulge between positions 10 and 11 when the small RNA is paired with the miRNA recognition site. The resulting synthetic small RNA, a cleavage blocker, can reduce endogenous miRNA binding and thus block cleavage of a protected miRNA target site enhancing the expression of a target protein.

A recombinant DNA construct designed for producing a modified messenger RNA for the protein where the native miRNA recognition site is modified to be resistant to the binding of cognate miRNA which regulates the native gene can also be used to express protein from heterologous messenger RNA that is no longer modulated by the native miRNA.

The construction and description of such recombinant DNA constructs is disclosed in US Patent Application Publication US 2009/0070898 A1, and U.S. application Ser. No. 61/077,244, filed Jul. 1, 2008, both of which are incorporated herein by reference.

The activity of a miRNA which down-regulates an endogenous protein is enhanced by enhancing the expression of the miRNA or by enhancing the ability of the miRNA to bind an RNA encoding the target protein. A recombinant DNA encoding an RNA encoding the miRNA or a miRNA-sensitive messenger RNA encoding the protein in which a miRNA binding site is added are designed to enhance miRNA activity resulting in enhanced suppression of the target mRNA and cognate protein. Recombinant DNA encoding an RNA encoding a miRNA, or a miRNA-sensitive RNA are designed using methods disclosed in US Patent Application Publication US 2009/0070898 A1.

Some, if not many, miRNAs modulate the expression of multiple proteins or biochemical pathways. Transgenic plants can be provided with enhanced traits not so much from the suppression or enhancement of the expression of a particular protein, as from change of enzyme activity in a pathway by modulating the level of a miRNA. Thus, aspects of this invention are achieved by enhanced miRNA activity resulting from use in transgenic plant cells of recombinant DNA constructs that produce an enhanced level of a miRNA. Other aspects of this invention are achieved by reduced miRNA activity resulting from use in transgenic plant cells of recombinant DNA constructs that produce a reduced level or activity of a miRNA.

Recombinant DNA constructs are assembled using methods well known to persons of ordinary skill in the art and typically comprise a promoter operably linked to DNA that is transcribed to RNA, e.g. messenger RNA for encoding a protein or RNA that is designed to effect a gene suppression pathway. Other construct components may include additional regulatory elements, such as 5′ leaders and introns for enhancing transcription, 3′ untranslated regions (such as polyadenylation signals and sites), DNA for transit or signal peptides. Numerous promoters that are active in plant cells have been described in the literature and patents and known to those skilled in the art. A wide range of such elements and their use in the design of recombinant DNA constructs is especially well disclosed in U.S. Pat. No. 5,977,441, incorporated herein by reference.

Transgenic plants comprising or derived from plant cells of this invention transformed with recombinant DNA can be further enhanced with stacked traits, e.g. a crop plant having an enhanced trait resulting from expression of DNA disclosed herein in combination with herbicide and/or pest resistance traits. For example, genes of the current invention can be stacked with other traits of agronomic interest, such as a trait providing herbicide resistance, or insect resistance, such as using a gene from Bacillus thuringensis to provide resistance against lepidopteran, coliopteran, homopteran, hemiopteran, and other insects. Herbicides for which transgenic plant tolerance has been demonstrated and the method of the present invention can be applied include, but are not limited to, glyphosate, dicamba, glufosinate, sulfonylurea, bromoxynil and norflurazon herbicides. Polynucleotide molecules encoding proteins involved in herbicide tolerance are well-known in the art and include, but are not limited to, a polynucleotide molecule encoding 5-enolpyruvylshikimate-3-phosphate synthase (EPSPS) disclosed in U.S. Pat. Nos. 5,094,945; 5,627,061; 5,633,435 and 6,040,497 for imparting glyphosate tolerance; polynucleotide molecules encoding a glyphosate oxidoreductase (GOX) disclosed in U.S. Pat. No. 5,463,175 and a glyphosate-N-acetyl transferase (GAT) disclosed in US Patent Application Publication 2003/0083480 A1 also for imparting glyphosate tolerance; dicamba monooxygenase disclosed in US Patent Application Publication 2003/0135879 A1 for imparting dicamba tolerance; a polynucleotide molecule encoding bromoxynil nitrilase (Bxn) disclosed in U.S. Pat. No. 4,810,648 for imparting bromoxynil tolerance; a polynucleotide molecule encoding phytoene desaturase (crtI) described in Misawa et al, (1993) Plant J. 4:833-840 and in Misawa et al, (1994) Plant J. 6:481-489 for norflurazon tolerance; a polynucleotide molecule encoding acetohydroxyacid synthase (AHAS, aka ALS) described in Sathasiivan et al. (1990) Nucl. Acids Res. 18:2188-2193 for imparting tolerance to sulfonylurea herbicides; polynucleotide molecules known as bar genes disclosed in DeBlock, et al. (1987) EMBO J. 6:2513-2519 for imparting glufosinate and bialaphos tolerance; polynucleotide molecules disclosed in US Patent Application Publication 2003/010609 A1 for imparting N-amino methyl phosphonic acid tolerance; polynucleotide molecules disclosed in U.S. Pat. No. 6,107,549 for impartinig pyridine herbicide resistance; molecules and methods for imparting tolerance to multiple herbicides such as glyphosate, atrazine, ALS inhibitors, isoxoflutole and glufosinate herbicides are disclosed in U.S. Pat. No. 6,376,754 and US Patent Application Publication 2002/0112260. Molecules and methods for imparting insect/nematode/virus resistance are disclosed in U.S. Pat. Nos. 5,250,515; 5,880,275; 6,506,599; 5,986,175 and US Patent Application Publication 2003/0150017 A1.

### Plant Cell Transformation Methods

Numerous methods for transforming chromosomes in a plant cell nucleus with recombinant DNA are known in the art and are used in methods of preparing a transgenic plant cell nucleus cell, and plant. Two effective methods for such transformation are Agrobacterium-mediated transformation and microprojectile bombardment. Microprojectile bombardment methods are illustrated in U.S. Pat. No. 5,015,580 (soybean); U.S. Pat. No. 5,550,318 (corn); U.S. Pat. No. 5,538,880 (corn); U.S. Pat. No. 5,914,451 (soybean); U.S. Pat. No. 6,160,208 (corn); U.S. Pat. No. 6,399,861 (corn); U.S. Pat. No. 6,153,812 (wheat) and U.S. Pat. No. 6,365,807 (rice) and Agrobacterium-mediated transformation is described in U.S. Pat. No. 5,159,135 (cotton); U.S. Pat. No. 5,824,877 (soybean); U.S. Pat. No. 5,463,174 (canola); U.S. Pat. No. 5,591,616 (corn); U.S. Pat. No. 6,384,301 (soybean), U.S. Pat. No. 7,026,528 (wheat) U.S. Pat. No. 6,329,571 (rice), and US Patent Application Publication 2001/0042257 A1 (sugar beet), all of which are incorporated herein by reference for enabling the production of transgenic plants. Transformation of plant material is practiced in tissue culture on a nutrient media, i.e. a mixture of nutrients that will allow cells to grow in vitro. Recipient cell targets include, but are not limited to, meristem cells, hypocotyls, calli, immature embryos and gametic cells such as microspores, pollen, sperm and egg cells. Callus may be initiated from tissue sources including, but not limited to, immature embryos, hypocotyls, seedling apical meristems, microspores and the like. Cells containing a transgenic nucleus are grown into transgenic plants.

In addition to direct transformation of a plant material with a recombinant DNA, a transgenic plant cell nucleus can be prepared by crossing a first plant having cells with a transgenic nucleus with recombinant DNA with a second plant lacking the trangenci nucleus. For example, recombinant DNA can be introduced into a nucleus from a first plant line that is amenable to transformation to transgenic nucleus in cells that are grown into a transgenic plant which can be crossed with a second plant line to introgress the recombinant DNA into the second plant line. A transgenic plant with recombinant DNA providing an enhanced trait, e.g. enhanced yield, can be crossed with transgenic plant line having other recombinant DNA that confers another trait, for example herbicide resistance or pest resistance, to produce progeny plants having recombinant DNA that confers both traits. Typically, in such breeding for combining traits the transgenic plant donating the additional trait is a male line and the transgenic plant carrying the base traits is the female line. The progeny of this cross will segregate such that some of the plants will carry the DNA for both parental traits and some will carry DNA for one parental trait; such plants can be identified by markers associated with parental recombinant DNA, e.g. marker identification by analysis for recombinant DNA or, in the case where a selectable marker is linked to the recombinant, by application of the selecting agent such as a herbicide for use with a herbicide tolerance marker, or by selection for the enhanced trait. Progeny plants carrying DNA for both parental traits can be crossed back into the female parent line multiple times, for example usually 6 to 8 generations, to produce a progeny plant with substantially the same genotype as one original transgenic parental line but for the recombinant DNA of the other transgenic parental line

In the practice of transformation DNA is typically introduced into only a small percentage of target plant cells in any one transformation experiment. Marker genes are used to provide an efficient system for identification of those cells that are stably transformed by receiving and integrating a recombinant DNA molecule into their genomes. Preferred marker genes provide selective markers which confer resistance to a selective agent, such as an antibiotic or a herbicide. Any of the herbicides to which plants of this invention may be resistant are useful agents for selective markers. Potentially transformed cells are exposed to the selective agent. In the population of surviving cells will be those cells where, generally, the resistance-conferring gene is integrated and expressed at sufficient levels to permit cell survival. Cells may be tested further to confirm stable integration of the exogenous DNA. Commonly used selective marker genes include those conferring resistance to antibiotics such as kanamycin and paromomycin (nptII), hygromycin B (aph IV), spectinomycin (aadA) and gentamycin (aac3 and aacC4) or resistance to herbicides such as glufosinate (bar or pat), dicamba (DMO) and glyphosate (aroA or EPSPS). Examples of such selectable markers are illustrated in U.S. Pat. Nos. 5,550,318; 5,633,435; 5,780,708 and 6,118,047. Selectable markers which provide an ability to visually identify transformants can also be employed, for example, a gene expressing a colored or fluorescent protein such as a luciferase or green fluorescent protein (GFP) or a gene expressing a beta-glucuronidase or uidA gene (GUS) for which various chromogenic substrates are known.

Plant cells that survive exposure to the selective agent, or plant cells that have been scored positive in a screening assay, may be cultured in regeneration media and allowed to mature into plants. Developing plantlets regenerated from transformed plant cells can be transferred to plant growth mix, and hardened off, for example, in an environmentally controlled chamber at about 85% relative humidity, 600 ppm CO₂, and 25-250 microeinsteins m⁻²s⁻¹ of light, prior to transfer to a greenhouse or growth chamber for maturation. Plants are regenerated from about 6 weeks to 10 months after a transformant is identified, depending on the initial tissue, and plant species. Plants may be pollinated using conventional plant breeding methods known to those of skill in the art and seed produced, for example self-pollination is commonly used with transgenic corn. The regenerated transformed plant or its progeny seed or plants can be tested for expression of the recombinant DNA and selected for the presence of enhanced agronomic trait.

### Transgenic Plants and Seeds

Transgenic plants derived from transgenic plant cells having a transgenic nucleus of this invention are grown to generate transgenic plants having an enhanced trait as compared to a control plant and produce transgenic seed and haploid pollen of this invention. Such plants with enhanced traits are identified by selection of transformed plants or progeny seed for the enhanced trait. For efficiency a selection method is designed to evaluate multiple transgenic plants (events) comprising the recombinant DNA, for example multiple plants from 2 to 20 or more transgenic events. Transgenic plants grown from transgenic seed provided herein demonstrate improved agronomic traits that contribute to increased yield or other trait that provides increased plant value, including, for example, improved seed quality. Of particular interest are plants having enhanced water use efficiency, enhanced cold tolerance, increased yield, enhanced nitrogen use efficiency, enhanced seed protein and enhanced seed oil.

Table 1 provides a list of protein encoding DNA (“genes”) and miRNAs that are useful in the production of transgenic plants with enhanced agronomic trait. The elements of Table 1 are described by reference to:

“PEP SEQ ID NO” identifies an amino acid sequence from SEQ ID NO: 16 to 21.

“NUC SEQ ID NO” identifies a nucleotide sequence from SEQ ID NO:1 to 15.

“EXP SEQ ID NO” identifies an exemplary DNA sequence of SEQ ID NOs: 1128 to 1140 used to produce a desired change in an identified miRNA or protein.

“Gene/miRNA ID” refers to an arbitrary identifier.

“Gene/miRNA Name” denotes a common name for the protein or miRNA encoded by the recombinant DNA.

“Annotation” refers to a description of the top hit protein obtained from an amino acid sequence query of each PEP SEQ ID NO to GENBANK database of the National Center for Biotechnology Information (ncbi).

TABLE 1

| NUC | PEP | EXP | | | |
|---|---|---|---|---|---|
| SEQ | SEQ | SEQ | | | |
| ID | ID | ID | | Gene/miRNA | |
| NO | NO | NO | Gene/miRNA ID | Name | Annotation |
| 1 | | 1128 | Mnom000623 | mir319 | |
| 2 | | | Mnom000624-Mnom000625 | mir171 | |
| 3 | 16 | 1129 | Mnom000630-Mnom000632 | ZmGw2 | RING-type E3 ubiquitin |
| | | | | | ligase [Oryza sativa |
| | | | | | (indica cultivar-group)] |
| 4 | 17 | 1130 | Mnom000891 | ZmHB4 | Homeodomain |
| | | | | | transcription factor |
| | | | | | HOX17 |
| 5 | 18 | 1131 | Mnom000894-Mnom000895 | ANR1 | MADS-box transcription |
| | | | | | factor 25 |
| 6 | 19 | 1132 | Mnom000896 | LG1 | LG1_MAIZE Protein |
| | | | | | LIGULELESS 1 |
| 7 | | 1133 | Mnom000897 | mir444 | |
| 8 | 20 | 1134 | Mnom000945, | SBP | squamosa promoter- |
| | | | Mnom001799-Mnom001803 | | binding protein-like 11 |
| 9 | | 1135 | Mnom001247-Mnom001248 | mir393 | |
| 10 | | 1136 | Mnom001249-Mnom001250 | mir398 | |
| 11 | | 1137 | Mnom001249-Mnom001250 | mir408 | |
| 12 | | 1138 | Mnom001249-Mnom001250 | mir528 | |
| 13 | | 1139 | Mnom001251-Mnom001252 | mir169g | |
| 14 | 21 | | Mnom001677 | IPS | 2-isopropylmalate |
| | | | | | synthase B, putative, |
| | | | | | expressed |
| 15 | | 1140 | Mnom001804-Mnom001805, | mir397 | |
| | | | Mnom001816-Mnom001817 | | |

Selection Methods for Transgenic Plants with Enhanced Agronomic Trait

Within a population of transgenic plants each regenerated from a plant cell having a nucleus with recombinant DNA many plants that survive to fertile transgenic plants that produce seeds and progeny plants will not exhibit an enhanced agronomic trait. Selection from the population is necessary to identify one or more transgenic plant cells having a transgenic nucleus that can provide plants with the enhanced trait. Transgenic plants having enhanced traits are selected from populations of plants regenerated or derived from plant cells transformed as described herein by evaluating the plants in a variety of assays to detect an enhanced trait, e.g. enhanced water use efficiency, enhanced cold tolerance, increased yield, enhanced nitrogen use efficiency, enhanced seed protein and enhanced seed oil. These assays also may take many forms including, but not limited to, direct screening for the trait in a greenhouse or field trial or by screening for a surrogate trait. Such analyses can be directed to detecting changes in the chemical composition, biomass, physiological properties, morphology of the plant. Changes in chemical compositions such as nutritional composition of grain can be detected by analysis of the seed composition and content of protein, free amino acids, oil, free fatty acids, starch or tocopherols. Changes in biomass characteristics can be made on greenhouse or field grown plants and can include plant height, stem diameter, root and shoot dry weights; and, for corn plants, ear length and diameter. Changes in physiological properties can be identified by evaluating responses to stress conditions, for example assays using imposed stress conditions such as water deficit, nitrogen deficiency, cold growing conditions, pathogen or insect attack or light deficiency, or increased plant density. Changes in morphology can be measured by visual observation of tendency of a transformed plant with an enhanced agronomic trait to also appear to be a normal plant as compared to changes toward bushy, taller, thicker, narrower leaves, striped leaves, knotted trait, chlorosis, albino, anthocyanin production, or altered tassels, ears or roots. Other selection properties include days to pollen shed, days to silking, leaf extension rate, chlorophyll content, leaf temperature, stand, seedling vigor, internode length, plant height, leaf number, leaf area, tillering, brace roots, stay green, stalk lodging, root lodging, plant health, barreness/prolificacy, green snap, and pest resistance. In addition, phenotypic characteristics of harvested grain may be evaluated, including number of kernels per row on the ear, number of rows of kernels on the ear, kernel abortion, kernel weight, kernel size, kernel density and physical grain quality.

Assays for screening for a desired trait are readily designed by those practicing in the art. The following illustrates useful screening assays for corn traits using hybrid corn plants. The assays can be readily adapted for screening other plants such as canola, cotton and soybean either as hybrids or inbreds.

Transgenic corn plants having nitrogen use efficiency are identified by screening in fields with three levels of nitrogen (N) fertilizer being applied, e.g. low level (0 N), medium level (80 lb/ac) and high level (180 lb/ac). Plants with enhanced nitrogen use efficiency provide higher yield as compared to control plants.

Transgenic corn plants having enhanced yield are identified by screening using progeny of the transgenic plants over multiple locations with plants grown under optimal production management practices and maximum weed and pest control. A useful target for improved yield is a 5% to 10% increase in yield as compared to yield produced by plants grown from seed for a control plant. Selection methods may be applied in multiple and diverse geographic locations, for example up to 16 or more locations, over one or more planting seasons, for example at least two planting seasons, to statistically distinguish yield improvement from natural environmental effects.

Transgenic corn plants having enhanced water use efficiency are identified by screening plants in an assay where water is withheld for period to induce stress followed by watering to revive the plants. For example, a useful selection process imposes 3 drought/re-water cycles on plants over a total period of 15 days after an initial stress free growth period of 11 days. Each cycle consists of 5 days, with no water being applied for the first four days and a water quenching on the 5th day of the cycle. The primary phenotypes analyzed by the selection method are the changes in plant growth rate as determined by height and biomass during a vegetative drought treatment.

Transgenic corn plants having enhanced cold tolerance are identified by screening plants in a cold germination assay and/or a cold tolerance field trial. In a cold germination assay trays of transgenic and control seeds are placed in a growth chamber at 9.7° C. for 24 days (no light). Seeds having higher germination rates as compared to the control are identified as having enhanced cold tolerance. In a cold tolerance field trial plants with enhanced cold tolerance are identified from field planting at an earlier date than conventional Spring planting for the field location. For example, seeds are planted into the ground around two weeks before local farmers begin to plant corn so that a significant cold stress is exerted onto the crop, named as cold treatment. Seeds also are planted under local optimal planting conditions such that the crop has little or no exposure to cold condition, named as normal treatment. At each location, seeds are planted under both cold and normal conditions preferably with multiple repetitions per treatment.

Transgenic corn plants having seeds with increased protein and/or oil levels are identified by analyzing progeny seed for protein and/or oil. Near-infrared transmittance spectrometry is a non-destructive, high-throughput method that is useful to determine the composition of a bulk seed sample for properties listed in table 2.

TABLE 2

| | |
|---|---|
| Typical sample(s): | Whole grain corn and soybean seeds |
| Typical analytical range: | Corn - moisture 5-15%, oil 5-20%, |
| | protein 5-30%, starch 50-75%, and density |
| | 1.0-1.3%. |
| | Soybean - moisture 5-15%, oil 15-25%, |
| | and protein 35-50%. |

Although the plant cells and methods of this invention can be applied to any plant cell, plant, seed or pollen, e.g. any fruit, vegetable, grass, tree or ornamental plant, the various aspects of the invention are preferably applied to corn, soybean, cotton, canola, alfalfa, wheat, rice, sugarcane, and sugarbeat plants. In many cases the invention is applied to corn plants that are inherently resistant to disease from the Mal de Rio Cuarto virus or the Puccina sorghi fungus or both.

The following examples are included to demonstrate aspects of the invention, those of skill in the art should, in light of the present disclosure, appreciate that many changes can be made in the specific aspects which are disclosed and still obtain a like or similar results without departing from the spirit and scope of the invention.

### Example 1

### Plant Expression Constructs

This example illustrates the construction of plasmids for transferring recombinant DNA into a plant cell nucleus that can be regenerated into transgenic plants.

### A. Plant Expression Constructs for Corn Transformation

A base corn transformation vector pMON93039, as set forth in SEQ ID NO:22, illustrated in Table 3, is fabricated for use in preparing recombinant DNA for Agrobacterium-mediated transformation into corn tissue.

TABLE 3

| | | | Coordinates of |
|---|---|---|---|
| Function | Name | Annotation | SEQ ID NO: 22 |
| Agrobacterium | B-AGRtu.right border | Agro right border sequence, | 11364-11720 |
| T-DNA transfer | | essential for transfer of T-DNA. | |
| Gene of interest | E-Os.Act1 | Upstream promoter region of the | 19-775 |
| expression | | rice actin 1 gene | |
| cassette | E-CaMV.35S.2xA1- | Duplicated35S A1-B3 domain | 788-1120 |
| | B3 | without TATA box | |
| | P-Os.Act1 | Promoter region of the rice actin 1 | 1125-1204 |
| | | gene | |
| | L-Ta.Lhcb1 | 5′ untranslated leader of wheat | 1210-1270 |
| | | major chlorophyll a/b binding | |
| | | protein | |
| | I-Os.Act1 | First intron and flanking UTR exon | 1287-1766 |
| | | sequences from the rice actin 1 | |
| | | gene | |
| | T-St.Pis4 | 3′ non-translated region of the | 1838-2780 |
| | | potato proteinase inhibitor II gene | |
| | | which functions to direct | |
| | | polyadenylation of the mRNA | |
| Plant selectable | P-Os.Act1 | Promoter from the rice actin 1 gene | 2830-3670 |
| marker | L-Os.Act1 | First exon of the rice actin 1 gene | 3671-3750 |
| expression | I-Os.Act1 | First intron and flanking UTR exon | 3751-4228 |
| cassette | | sequences from the rice actin 1 | |
| | | gene | |
| | TS-At.ShkG-CTP2 | Transit peptide region of | 4238-4465 |
| | | Arabidopsis EPSPS | |
| | CR-AGRtu.aroA- | Coding region for bacterial strain | 4466-5833 |
| | CP4.nat | CP4 native aroA gene. | |
| | T-AGRtu.nos | A 3′ non-translated region of the | 5849-6101 |
| | | nopaline synthase gene of | |
| | | Agrobacterium tumefaciens Ti | |
| | | plasmid which functions to direct | |
| | | polyadenylation of the mRNA. | |
| Agrobacterium | B-AGRtu.left border | Agro left border sequence, essential | 6168-6609 |
| T-DNA transfer | | for transfer of T-DNA. | |
| Maintenance in | OR-Ec.oriV-RK2 | The vegetative origin of replication | 6696-7092 |
| E. coli | | from plasmid RK2. | |
| | CR-Ec.rop | Coding region for repressor of | 8601-8792 |
| | | primer from the ColE1 plasmid. | |
| | | Expression of this gene product | |
| | | interferes with primer binding at the | |
| | | origin of replication, keeping | |
| | | plasmid copy number low. | |
| | OR-Ec.ori-ColE1 | The minimal origin of replication | 9220-9808 |
| | | from the E. coli plasmid ColE1. | |
| | P-Ec.aadA-SPC/STR | Promoter for Tn7 | 10339-10380 |
| | | adenylyltransferase (AAD(3″)) | |
| | CR-Ec.aadA- | Coding region for Tn7 | 10381-11169 |
| | SPC/STR | adenylyltransferase (AAD(3″)) | |
| | | conferring spectinomycin and | |
| | | streptomycin resistance. | |
| | T-Ec.aadA-SPC/STR | 3′ UTR from the Tn7 | 11170-11227 |
| | | adenylyltransferase (AAD(3″)) | |
| | | gene of E. coli. | |

To construct transformation vectors for expressing a protein identified in Table 1, primers for PCR amplification of the protein coding nucleotides are designed at or near the start and stop codons of the coding sequence, in order to eliminate most of the 5′ and 3′ untranslated regions. The protein coding nucleotides are inserted into the base vector in the gene of interest expression cassette at an insertion site, i.e. between the intron element (coordinates 1287-1766) and the polyadenylation element (coordinates 1838-2780).

To construct transformation vectors for suppressing a protein identified in Table 1, the amplified protein coding nucleotides are assembled in a sense and antisense arrangement and inserted into the base vector at the insertion site in the gene of interest expression cassette to provide transcribed RNA that will form a double-stranded RNA for RNA interference suppression of the protein. In the embodiments of this invention the proteins that are suppressed are ZmGw2, ZmHB4, LG1, and IPS.

### B. Plant Expression Constructs for Soy and Canola Transformation

Vectors for use in transformation of soybean and canola tissue are prepared having the elements of expression vector pMON82053 (SEQ ID NO: 23) as shown in Table 4 below.

TABLE 4

| | | | Coordinates of |
|---|---|---|---|
| Function | Name | Annotation | SEQ ID NO: 23 |
| Agrobacterium T- | B-AGRtu.left border | Agro left border sequence, essential for | 6144-6585 |
| DNA transfer | | transfer of T-DNA. | |
| Plant selectable | P-At.Act7 | Promoter from the Arabidopsis actin 7 gene | 6624-7861 |
| marker expression | L-At.Act7 | 5′UTR of Arabidopsis Act7 gene | |
| cassette | I-At.Act7 | Intron from the Arabidopsis actin7 gene | |
| | TS-At.ShkG-CTP2 | Transit peptide region of Arabidopsis | 7864-8091 |
| | | EPSPS | |
| | CR-AGRtu.aroA- | Synthetic CP4 coding region with dicot | 8092-9459 |
| | CP4.nno_At | preferred codon usage. | |
| | T-AGRtu.nos | A 3′ non-translated region of the nopaline | 9466-9718 |
| | | synthase gene of Agrobacterium | |
| | | tumefaciens Ti plasmid which functions to | |
| | | direct polyadenylation of the mRNA. | |
| Gene of interest | P-CaMV.35S-enh | Promoter for 35S RNA from CaMV | 1-613 |
| expression cassette | | containing a duplication of the −90 to −350 | |
| | | region. | |
| | T-Gb.E6-3b | 3′ untranslated region from the fiber protein | 688-1002 |
| | | E6 gene of sea-island cotton. | |
| Agrobacterium T- | B-AGRtu.right | Agro right border sequence, essential for | 1033-1389 |
| DNA transfer | border | transfer of T-DNA. | |
| Maintenance in E. coli | OR-Ec.oriV-RK2 | The vegetative origin of replication from | 5661-6057 |
| | | plasmid RK2. | |
| | CR-Ec.rop | Coding region for repressor of primer from | 3961-4152 |
| | | the ColE1 plasmid. Expression of this gene | |
| | | product interferes with primer binding at the | |
| | | origin of replication, keeping plasmid copy | |
| | | number low. | |
| | OR-Ec.ori-ColE1 | The minimal origin of replication from the | 2945-3533 |
| | | E. coli plasmid ColE1. | |
| | P-Ec.aadA-SPC/STR | Promoter for Tn7 adenylyltransferase | 2373-2414 |
| | | (AAD(3″)) | |
| | CR-Ec.aadA- | Coding region for Tn7 adenylyltransferase | 1584-2372 |
| | SPC/STR | (AAD(3″)) conferring spectinomycin and | |
| | | streptomycin resistance. | |
| | T-Ec.aadA-SPC/STR | 3′ UTR from the Tn7 adenylyltransferase | 1526-1583 |
| | | (AAD(3″)) gene of E. coli. | |

To construct transformation vectors for expressing a protein identified Table 1 primers for PCR amplification of the protein coding nucleotides are designed at or near the start and stop codons of the coding sequence, in order to eliminate most of the 5′ and 3′ untranslated regions. The protein coding nucleotides are inserted into the base vector in the gene of interest expression cassette at an insertion site, i.e. between the promoter element (coordinates 1-613) and the polyadenylation element (coordinates 688-1002).

To construct transformation vectors for suppressing a protein identified Table 1 the amplified protein coding nucleotides are assembled in a sense and antisense arrangement and inserted into the base vector at the insertion site in the gene of interest expression cassette to provide transcribed RNA that will form a double-stranded RNA for RNA interference suppression of the protein. In the embodiments of this invention the proteins that are suppressed are ZmGw2, ZmHB4, LG1, and IPS.

### C. Cotton Transformation Vector

Plasmids for use in transformation of cotton tissue are prepared with elements of expression vector pMON99053 (SEQ ID NO: 24) as shown in Table 5 below.

TABLE 5

| | | | Coordinates of SEQ ID NO: |
|---|---|---|---|
| Function | Name | Annotation | 24 |
| Agrobacterium T- | B-AGRtu.right border | Agro right border sequence, | 1-357 |
| DNA transfer | | essential for transfer of T-DNA. | |
| Gene of interest | Exp-CaMV.35S- | Enhanced version of the 35S RNA | 388-1091 |
| expression | enh + Ph.DnaK | promoter from CaMV plus the | |
| cassette | | petunia hsp70 5′ untranslated region | |
| | T-Ps.RbcS2-E9 | The 3′ non-translated region of the | 1165-1797 |
| | | pea RbcS2 gene which functions to | |
| | | direct polyadenylation of the | |
| | | mRNA. | |
| Plant selectable | Exp-CaMV.35S | Promoter and 5′ untranslated region | 1828-2151 |
| marker expression | | from the 35S RNA of CaMV | |
| cassette | CR-Ec.nptII-Tn5 | Coding region for neomycin | 2185-2979 |
| | | phosphotransferase gene from | |
| | | transposon Tn5 which confers | |
| | | resistance to neomycin and | |
| | | kanamycin. | |
| | T-AGRtu.nos | A 3′ non-translated region of the | 3011-3263 |
| | | nopaline synthase gene of | |
| | | Agrobacterium tumefaciens Ti | |
| | | plasmid which functions to direct | |
| | | polyadenylation of the mRNA. | |
| Agrobacterium T- | B-AGRtu.left border | Agro left border sequence, essential | 3309-3750 |
| DNA transfer | | for transfer of T-DNA. | |
| Maintenance in E. coli | OR-Ec.oriV-RK2 | The vegetative origin of replication | 3837-4233 |
| | | from plasmid RK2. | |
| | CR-Ec.rop | Coding region for repressor of | 5742-5933 |
| | | primer from the ColE1 plasmid. | |
| | | Expression of this gene product | |
| | | interferes with primer binding at the | |
| | | origin of replication, keeping | |
| | | plasmid copy number low. | |
| | OR-Ec.ori-ColE1 | The minimal origin of replication | 6361-6949 |
| | | from the E. coli plasmid ColE1. | |
| | P-Ec.aadA-SPC/STR | Promoter for Tn7 | 7480-7521 |
| | | adenylyltransferase (AAD(3″)) | |
| | CR-Ec.aadA-SPC/STR | Coding region for Tn7 | 7522-8310 |
| | | adenylyltransferase (AAD(3″)) | |
| | | conferring spectinomycin and | |
| | | streptomycin resistance. | |
| | T-Ec.aadA-SPC/STR | 3′ UTR from the Tn7 | 8311-8368 |
| | | adenylyltransferase (AAD(3″)) gene | |
| | | of E. coli. | |

To construct transformation vectors for expressing a protein identified Table 1 primers for PCR amplification of the protein coding nucleotides are designed at or near the start and stop codons of the coding sequence, in order to eliminate most of the 5′ and 3′ untranslated regions. The protein coding nucleotides are inserted into the base vector in the gene of interest expression cassette at an insertion site, i.e. between the promoter element (coordinates 388-1091) and the polyadenylation element (coordinates 1165-1791).

To construct transformation vectors for suppressing a protein identified Table 1 the amplified protein coding nucleotides are assembled in a sense and antisense arrangement and inserted into the base vector at the insertion site in the gene of interest expression cassette to provide transcribed RNA that will form a double-stranded RNA for RNA interference suppression of the protein. In the embodiments of this invention the proteins that are suppressed are ZmGw2, ZmHB4, LG1, and IPS.

### Example 2

### Corn Transformation

This example illustrates transformation methods useful in producing a transgenic nucleus in a corn plant cell, and the plants, seeds and pollen produced from a transgenic cell with such a nucleus having an enhanced trait, i.e. enhanced water use efficiency, enhanced cold tolerance, increased yield, enhanced nitrogen use efficiency, enhanced seed protein and enhanced seed oil. A plasmid vector is prepared by cloning the DNA of SEQ ID NO:3 into the gene of interest expression cassette in the base vector for use in corn transformation of corn tissue provided in Example 1, Table 3.

For Agrobacterium-mediated transformation of corn embryo cells corn plants of a readily transformable line are grown in the greenhouse and ears are harvested when the embryos are 1.5 to 2.0 mm in length. Ears are surface sterilized by spraying or soaking the ears in 80% ethanol, followed by air drying Immature embryos are isolated from individual kernels on surface sterilized ears. Prior to inoculation of maize cells, Agrobacterium cells are grown overnight at room temperature Immature maize embryo cells are inoculated with Agrobacterium shortly after excision, and incubated at room temperature with Agrobacterium for 5-20 minutes Immature embryo plant cells are then co-cultured with Agrobacterium for 1 to 3 days at 23° C. in the dark. Co-cultured embryos are transferred to selection media and cultured for approximately two weeks to allow embryogenic callus to develop. Embryogenic callus is transferred to culture medium containing 100 mg/L paromomycin and subcultured at about two week intervals. Transformed plant cells are recovered 6 to 8 weeks after initiation of selection.

For Agrobacterium-mediated transformation of maize callus immature embryos are cultured for approximately 8-21 days after excision to allow callus to develop. Callus is then incubated for about 30 minutes at room temperature with the Agrobacterium suspension, followed by removal of the liquid by aspiration. The callus and Agrobacterium are co-cultured without selection for 3-6 days followed by selection on paromomycin for approximately 6 weeks, with biweekly transfers to fresh media. Paromomycin resistant calli are identified about 6-8 weeks after initiation of selection.

To regenerate transgenic corn plants a callus of transgenic plant cells resulting from transformation and selection is placed on media to initiate shoot development into plantlets which are transferred to potting soil for initial growth in a growth chamber at 26° C. followed by a mist bench before transplanting to 5 inch pots where plants are grown to maturity. The regenerated plants are self-fertilized and seed is harvested for use in one or more methods to select seeds, seedlings or progeny second generation transgenic plants (R2 plants) or hybrids, e.g. by selecting transgenic plants exhibiting an enhanced trait as compared to a control plant.

The above process is repeated to produce multiple events of transgenic corn plant cells that are transformed with recombinant DNA from each of the genes identified in Table 1. Events are designed to produce in the transgenic cells one of the proteins identified in Table 1, except the proteins of SEQ ID NOs:16, 17, 19, and 21 which are suppressed. Progeny transgenic plants and seed of the transformed plant cells are screened for enhanced water use efficiency, enhanced cold tolerance, increased yield, enhanced nitrogen use efficiency, enhanced seed protein and enhanced seed oil. From each group of multiple events of transgenic plants with a specific recombinant DNA from Table 1 the event that produces the greatest enhancement in yield, water use efficiency, nitrogen use efficiency, enhanced cold tolerance, enhanced seed protein and enhanced seed oil is identified and progeny seed is selected for commercial development.

### Example 3

### Soybean Transformation

This example illustrates plant transformation useful in producing a transgenic nucleus in a soybean plant cell, and the plants, seeds and pollen produced from a transgenic cell with such a nucleus having an enhanced trait, i.e. enhanced water use efficiency, enhanced cold tolerance, increased yield, enhanced nitrogen use efficiency, enhanced seed protein and enhanced seed oil.

For Agrobacterium mediated transformation, soybean seeds are imbided overnight and the meristem explants excised. The explants are placed in a wounding vessel. Soybean explants and induced Agrobacterium cells from a strain containing plasmid DNA with the gene of interest cassette and a plant selectable marker cassette are mixed no later than 14 hours from the time of initiation of seed imbibition, and wounded using sonication. Following wounding, explants are placed in co-culture for 2-5 days at which point they are transferred to selection media for 6-8 weeks to allow selection and growth of transgenic shoots. Resistant shoots are harvested approximately 6-8 weeks and placed into selective rooting media for 2-3 weeks. Shoots producing roots are transferred to the greenhouse and potted in soil. Shoots that remain healthy on selection, but do not produce roots are transferred to non-selective rooting media for an additional two weeks. Roots from any shoots that produce roots off selection are tested for expression of the plant selectable marker before they are transferred to the greenhouse and potted in soil.

The above process is repeated to produce multiple events of transgenic soybean plant cells that are transformed with recombinant DNA from each of the genes identified in Table 1. Events are designed to produce in the transgenic cells one of the proteins identified in Table 1, except the proteins of SEQ ID NOs:16, 17, 19, and 21 which are suppressed. Progeny transgenic plants and seed of the transformed plant cells are screened for enhanced water use efficiency, enhanced cold tolerance, increased yield, enhanced seed protein and enhanced seed oil. From each group of multiple events of transgenic plants with a specific recombinant DNA from Table 1 the event that produces the greatest enhancement in yield, water use efficiency, nitrogen use efficiency, enhanced cold tolerance, enhanced seed protein and enhanced seed oil is identified and progeny seed is selected for commercial development.

### Example 4

### Cotton Transgenic Plants with Enhanced Agronomic Traits

This example illustrates plant transformation useful in producing a transgenic nucleus in a cotton plant cell, and the plants, seeds and pollen produced from a transgenic cell with such a nucleus having an enhanced trait, i.e. enhanced water use efficiency, increased yield, enhanced nitrogen use efficiency and enhanced seed oil.

Transgenic cotton plants containing each recombinant DNA having a sequence of SEQ ID NO: 3-6, 8, and 14 are obtained by transforming with recombinant DNA from each of the genes identified in Table 1 using Agrobacterium-mediated transformation. The above process is repeated to produce multiple events of transgenic cotton plant cells that are transformed with recombinant DNA from each of the genes identified in Table 1. Events are designed to produce in the transgenic cells one of the proteins identified in Table 1, except the proteins of SEQ ID NOs:16, 17, 19, and 21 which are suppressed.

From each group of multiple events of transgenic plants with a specific recombinant DNA from Table 1 the event that produces the greatest enhancement in yield, water use efficiency, nitrogen use efficiency, enhanced cold tolerance, enhanced seed protein and enhanced seed oil is identified and progeny seed is selected for commercial development.

Progeny transgenic plants are selected from a population of transgenic cotton events under specified growing conditions and are compared with control cotton plants. Control cotton plants are substantially the same cotton genotype but without the recombinant DNA, for example, either a parental cotton plant of the same genotype that was not transformed with the identical recombinant DNA or a negative isoline of the transformed plant. Additionally, a commercial cotton cultivar adapted to the geographical region and cultivation conditions, i.e. cotton variety ST474, cotton variety FM 958, and cotton variety Siokra L-23, are used to compare the relative performance of the transgenic cotton plants containing the recombinant DNA.

Transgenic cotton plants with enhanced yield and water use efficiency are identified by growing under variable water conditions. Specific conditions for cotton include growing a first set of transgenic and control plants under “wet” conditions, i.e. irrigated in the range of 85 to 100 percent of evapotranspiration to provide leaf water potential of −14 to −18 bars, and growing a second set of transgenic and control plants under “dry” conditions, i.e. irrigated in the range of 40 to 60 percent of evapotranspiration to provide a leaf water potential of −21 to −25 bars. Pest control, such as weed and insect control is applied equally to both wet and dry treatments as needed. Data gathered during the trial includes weather records throughout the growing season including detailed records of rainfall; soil characterization information; any herbicide or insecticide applications; any gross agronomic differences observed such as leaf morphology, branching habit, leaf color, time to flowering, and fruiting pattern; plant height at various points during the trial; stand density; node and fruit number including node above white flower and node above crack boll measurements; and visual wilt scoring. Cotton boll samples are taken and analyzed for lint fraction and fiber quality. The cotton is harvested at the normal harvest timeframe for the trial area. Enhanced water use efficiency is indicated by increased yield, improved relative water content, enhanced leaf water potential, increased biomass, enhanced leaf extension rates, and improved fiber parameters.

### Example 5

### Canola Transformation

This example illustrates plant transformation useful in producing the transgenic canola plants of this invention and the production and identification of transgenic seed for transgenic canola having enhanced water use efficiency, enhanced cold tolerance, increased yield, enhanced nitrogen use efficiency, enhanced seed protein and enhanced seed oil.

Tissues from in vitro grown canola seedlings are prepared and inoculated with overnight-grown Agrobacterium cells containing plasmid DNA with the gene of interest cassette and a plant selectable marker cassette. Following co-cultivation with Agrobacterium, the infected tissues are allowed to grow on selection to promote growth of transgenic shoots, followed by growth of roots from the transgenic shoots. The selected plantlets are then transferred to the greenhouse and potted in soil. Molecular characterization are performed to confirm the presence of the gene of interest, and its expression in transgenic plants and progenies. Progeny transgenic plants are selected from a population of transgenic canola events under specified growing conditions and are compared with control canola plants. Control canola plants are substantially the same canola genotype but without the recombinant DNA, for example, either a parental canola plant of the same genotype that is not transformed with the identical recombinant DNA or a negative isoline of the transformed plant

Transgenic canola plant cells are transformed with recombinant DNA from each of the genes identified in Table 1. The above process is repeated to produce multiple events of transgenic soybean plant cells that are transformed with recombinant DNA from each of the genes identified in Table 1. Events are designed to produce in the transgenic cells one of the proteins identified in Table 1, except the proteins of SEQ ID NOs:16, 17, 19, and 21 which are suppressed. Progeny transgenic plants and seed of the transformed plant cells are screened for enhanced water use efficiency, enhanced cold tolerance, increased yield, enhanced seed protein and enhanced seed oil. From each group of multiple events of transgenic plants with a specific recombinant DNA from Table 1 the event that produces the greatest enhancement in yield, water use efficiency, nitrogen use efficiency, enhanced cold tolerance, enhanced seed protein and enhanced seed oil is identified and progeny seed is selected for commercial development.

### Example 6

### Identification of Homologous Proteins

This example illustrates the identification of homologs of proteins encoded by the DNA identified in Table 1 which is used to provide transgenic seed and plants having enhanced agronomic traits. From the sequence of the homologs, homologous DNA sequence can be identified for preparing additional transgenic seeds and plants of this invention with enhanced agronomic traits.

An “All Protein Database” was constructed of known protein sequences using a proprietary sequence database and the National Center for Biotechnology Information (NCBI) non-redundant amino acid database (nr.aa). For each organism from which a polynucleotide sequence provided herein was obtained, an “Organism Protein Database” was constructed of known protein sequences of the organism; it is a subset of the All Protein Database based on the NCBI taxonomy ID for the organism.

The All Protein Database was queried using amino acid sequences provided herein as SEQ ID NO: 16 through SEQ ID NO: 21 using NCBI “blastp” program with E-value cutoff of 1e-8. Up to 1000 top hits were kept, and separated by organism names. For each organism other than that of the query sequence, a list was kept for hits from the query organism itself with a more significant E-value than the best hit of the organism. The list contains likely duplicated genes of the polynucleotides provided herein, and is referred to as the Core List. Another list was kept for all the hits from each organism, sorted by E-value, and referred to as the Hit List.

The Organism Protein Database was queried using polypeptide sequences provided herein as SEQ ID NO: 16 through SEQ ID NO: 21 using NCBI “blastp” program with E-value cutoff of 1e-4. Up to 1000 top hits were kept. A BLAST searchable database is constructed based on these hits, and is referred to as “SubDB”. SubDB was queried with each sequence in the Hit List using NCBI “blastp” program with E-value cutoff of 1e-8. The hit with the best E-value was compared with the Core List from the corresponding organism. The hit was deemed a likely ortholog if it belongs to the Core List, otherwise it was deemed not a likely ortholog and there was no further search of sequences in the Hit List for the same organism. Homologs from a large number of distinct organisms were identified and are reported below in table 6 with the SEQ ID NO of the original protein query sequence and the identified homologs as [SEQ ID NO]: [Homolog SEQ ID NOs].

TABLE 6
16: 25 26 27 28 29 30 31 32 33 34 35 36 37 38 39 40 41 42 43 44 45 46 47 48 49 50 51 52
53 54 55 56 57 58
17: 75 77 79 80 82 91 92 93 96 98 100 101 107 117 128 142 143 145 162 170 179 183 201
206 209 213 222 244 249 278 280 335 336 337 338 339 340 341 342 343 344 345 384 415
423 424 425 426 427 446 457 458 471 477 497 499 530 557 569 620 621 622 678 679 686
806 825 852 854 855 856 857 874
18: 64 65 66 67 68 72 73 81 83 85 88 99 122 134 150 173 181 198 221 237 241 250 259 260
264 324 325 326 329 334 375 386 395 400 402 403 440 441 442 449 492 493 504 508 509
521 522 523 554 555 561 572 579 580 581 582 584 589 591 592 593 599 603 604 608 632
634 639 640 641 642 643 691 692 717 733 734 766 768 774 778 790 791 835 836 842 847
851 861 875
19: 78 105 131 174 193 202 220 223 304 305 306 307 308 309 310 312 313 314 315 316
317 318 319 320 405 489 498 529 558 741 746 747 748 749 750 751 752 753 754 755 756 822
20: 63 84 97 106 108 110 111 112 113 114 120 123 127 129 130 132 135 136 138 139 140
141 175 193 210 211 218 219 245 246 279 281 282 306 307 308 315 322 350 353 389 404
405 437 478 481 482 484 485 486 488 494 496 505 515 526 527 528 532 560 636 675 746
747 748 749 750 751 752 753 754 755 756 765 769 822 880 881
21: 59 60 61 62 69 70 71 74 76 86 87 89 90 94 95 102 103 104 109 115 116 118 119 121
124 125 126 133 137 144 146 147 148 149 151 152 153 154 155 156 157 158 159 160 161
163 164 165 166 167 168 169 171 172 176 177 178 180 182 184 185 186 187 188 189 190
191 192 194 195 196 197 199 200 203 204 205 207 208 212 214 215 216 217 224 225 226
227 228 229 230 231 232 233 234 235 236 238 239 240 242 243 247 248 251 252 253 254
255 256 257 258 261 262 263 265 266 267 268 269 270 271 272 273 274 275 276 277 283
284 285 286 287 288 289 290 291 292 293 294 295 296 297 298 299 300 301 302 303 311
321 323 327 328 330 331 332 333 346 347 348 349 351 352 354 355 356 357 358 359 360
361 362 363 364 365 366 367 368 369 370 371 372 373 374 376 377 378 379 380 381 382
383 385 387 388 390 391 392 393 394 396 397 398 399 401 406 407 408 409 410 411 412
413 414 416 417 418 419 420 421 422 428 429 430 431 432 433 434 435 436 438 439 443
444 445 447 448 450 451 452 453 454 455 456 459 460 461 462 463 464 465 466 467 468
469 470 472 473 474 475 476 479 480 483 487 490 491 495 500 501 502 503 506 507 510
511 512 513 514 516 517 518 519 520 524 525 531 533 534 535 536 537 538 539 540 541
542 543 544 545 546 547 548 549 550 551 552 553 556 559 562 563 564 565 566 567 568
570 571 573 574 575 576 577 578 583 585 586 587 588 590 594 595 596 597 598 600 601
602 605 606 607 609 610 611 612 613 614 615 616 617 618 619 623 624 625 626 627 628
629 630 631 633 635 637 638 644 645 646 647 648 649 650 651 652 653 654 655 656 657
658 659 660 661 662 663 664 665 666 667 668 669 670 671 672 673 674 676 677 680 681
682 683 684 685 687 688 689 690 693 694 695 696 697 698 699 700 701 702 703 704 705
706 707 708 709 710 711 712 713 714 715 716 718 719 720 721 722 723 724 725 726 727
728 729 730 731 732 735 736 737 738 739 740 742 743 744 745 757 758 759 760 761 762
763 764 767 770 771 772 773 775 776 777 779 780 781 782 783 784 785 786 787 788 789
792 793 794 795 796 797 798 799 800 801 802 803 804 805 807 808 809 810 811 812 813
814 815 816 817 818 819 820 821 823 824 826 827 828 829 830 831 832 833 834 837 838
839 840 841 843 844 845 846 848 849 850 853 858 859 860 862 863 864 865 866 867 868
869 870 871 872 873 876 877 878 879 882 883 884 885 886 887 888 889 890 891 892 893 894

### Example 7

### Identification of Homologous miRNAs

ESTs were compared with the miRNAs of table 1. If an EST had a homolog region with ≦3 mismatches to a miRNA of table 1, then a subsequence of the EST of ˜230 nt with the homolog region at 5′ or 3′ end was predicted for stable secondary structure using RNAfold in EMBOSS package. A sequence was identified as a homologous miRNA if the secondary structure is typical for a pre-miRNA foldback structure and the homolog region, which is the miRNA candidate, has the required complementary pairing with the opposite arm.

Due to potential errors in EST data, the predicted pre-miRNAs were compared with reference genomic sequence to validate the accuracy of the sequence. Homologs from a large number of distinct organisms were identified and are reported below in table 7 with the SEQ ID NO of the original miRNA query sequence and the identified homologs as [SEQ ID NO]: [Homolog SEQ ID NOs].

TABLE 7

```
  1: 895-922
  2: 923-960
  7: 961-993
  9: 994-1002
10: 1003-1036
11: 1037-1049
12: 1050-1054
13: 1055-1119
15: 1120-1126
```

Recombinant DNA constructs are prepared using the DNA encoding each of the identified protein and miRNA homologs and the constructs are used to prepare multiple events of transgenic corn, soybean, canola and cotton plants as illustrated in Examples 2-5. Plants are regenerated from the transformed plant cells and used to produce progeny plants and seed that are screened for enhanced water use efficiency, enhanced cold tolerance, increased yield, enhanced nitrogen use efficiency, enhanced seed protein and enhanced seed oil. From each group of multiple events of transgenic plants with a specific recombinant DNA for a homolog the event that produces the greatest enhancement in yield, water use efficiency, nitrogen use efficiency, enhanced cold tolerance, enhanced seed protein and enhanced seed oil is identified and progeny seed is selected for commercial development.

### Example 8

### Consensus Sequence

This example illustrates the identification of consensus amino acid sequence for the proteins and homologs encoded by DNA that is used to prepare the transgenic seed and plants of this invention having enhanced agronomic traits.

ClustalW program was selected for multiple sequence alignments of the amino acid sequence of SEQ ID NO: 16 and its homologs. Three major factors affecting the sequence alignments dramatically are (1) protein weight matrices; (2) gap open penalty; (3) gap extension penalty. Protein weight matrices available for ClustalW program include Blosum, Pam and Gonnet series. Those parameters with gap open penalty and gap extension penalty were extensively tested. On the basis of the test results, Blosum weight matrix, gap open penalty of 10 and gap extension penalty of 1 were chosen for multiple sequence alignment.

The consensus amino acid sequence can be used to identify DNA corresponding to the full scope of this invention that is useful in providing transgenic plants, for example corn and soybean plants with enhanced agronomic traits, for example improved nitrogen use efficiency, improved yield, improved water use efficiency and/or improved growth under cold stress, due to the expression in the plants of DNA suppressing a protein with amino acid sequence identical to the consensus amino acid sequence.

The SEQ ID NOs for the identified consensus sequences are reported in table 8 below and the full consensus sequences are provided in the attached sequence listing.

TABLE 8

| PEP SEQ ID NO | Consensus SEQ ID NO |
|---|---|
| 16 | 1127 |

### Example 9

### Identification of Amino Acid Domain by Pfam Analysis

This example illustrates the identification of domain and domain module by Pfam analysis.

The amino acid sequence of the expressed proteins that are shown to be associated with an enhanced trait were analyzed for Pfam protein family against the current Pfam collection of multiple sequence alignments and hidden Markov models using the HMMER software. The Pfam protein domains and modules for the proteins of SEQ ID NOs: 17-21 are shown in Tables 9, 10 and 11. The Hidden Markov model databases for the identified patent families are also available from the Pfam consortium (ftp.sanger.ac.uk/pub/databases/Pfam/) allowing identification of other homologous proteins and their cognate encoding DNA to enable the full breadth of the invention for a person of ordinary skill in the art. Certain proteins are identified by a single Pfam domain and others by multiple Pfam domains. The function of the identified Pfam domains in proteins providing an enhanced trait in plants was verified by searching identified homologs for the conservation of the identified Pfam domains. The score value for the identified Pfam domains in sequences from table 1 and the minimum score value for the Pfam domain between a protein from table 1 and its identified homologs are reported below in table 9.

TABLE 9

| PEP SEQ | Pfam domain | | | | Minimum | |
|---|---|---|---|---|---|---|
| ID NO | name | Begin | Stop | Score | Score | E-value |
| 17 | Homeobox | 76 | 130 | 68.5 | 39.3 | 2.50E−17 |
| 17 | HALZ | 131 | 175 | 81.9 | 25.0 | 2.20E−21 |
| 18 | SRF-TF | 9 | 59 | 99.2 | 50.5 | 1.50E−26 |
| 18 | K-box | 75 | 172 | 79.2 | 20.9 | 1.50E−20 |
| 19 | SBP | 184 | 263 | 180.8 | 41.4 | 3.90E−51 |
| 20 | SBP | 174 | 252 | 178.8 | 41.4 | 1.50E−50 |
| 21 | HMGL-like | 84 | 366 | 352.5 | −75.3 | 8.20E−103 |
| 21 | LeuA_dimer | 459 | 604 | 180.3 | −30.8 | 5.60E−51 |

TABLE 10

| PEP SEQ ID NO | Pfam Domain Module | Position |
|---|---|---|
| 17 | Homeobox::HALZ | 76-130::131-175 |
| 18 | SRF-TF::K-box | 9-59::75-172 |
| 19 | SBP | 184-263 |
| 20 | SBP | 174-252 |
| 21 | HMGL-like::LeuA_dimer | 84-366::459-604 |

TABLE 11

| Pfam | | | |
|---|---|---|---|
| domain | Accession | Gathering | |
| name | number | cutoff | Domain description |
| HALZ | PF02183.10 | 18.1000; | Homeobox associated leucine |
| | | | zipper |
| HMGL-like | PF00682.11 | −82.2000; | HMGL-like |
| Homeobox | PF00046.21 | −4.1000; | Homeobox domain |
| K-box | PF01486.9 | 0.0000; | K-box region |
| LeuA_dimer | PF08502.2 | −37.5000; | LeuA allosteric (dimerisation) |
| | | | domain |
| SBP | PF03110.6 | 25.0000; | SBP domain |
| SRF-TF | PF00319.10 | 11.0000; | SRF-type transcription factor |
| | | | (DNA-binding and dimerisation |
| | | | domain) |

### Example 10

### Enhanced Expression of Target Genes

This example illustrates monocot and dicot plant transformation to produce recombinant DNA constructs that are useful for stable integration into plant chromosomes in the nuclei of plant cells to provide transgenic plants having enhanced traits by enhancement of the expression of target genes.

Various recombinant DNA constructs for use in enhancing the expression of a protein in transgenic plants are constructed based on the nucleotide sequence of the gene for producing the protein that has the amino acid sequence of SEQ ID NOs: 18 and 20, where the DNA constructs are designed to express (a) an RNA that is a messenger RNA that is translated to the protein but does not have a native miRNA recognition site thereby allowing enhanced expression of the target gene, (b) an RNA that produces dsRNA that is targeted to a native miRNA or pre-miRNA that natively regulates the protein accumulation, thereby allowing enhanced expression of the target gene (c) an RNA that functions as a decoy for the native miRNA, thereby allowing enhanced expression of the target gene, and (d) an RNA that binds to a miRNA recognition site in the messenger RNA for the protein to interfere with miRNA regulation of the messenger RNA.

Each of the various types of recombinant DNA construct is used in transformation of a corn cell using the vector and method of Example 2 to produce multiple events of transgenic corn cell that are each regenerated into transgenic corn plants that are screened to identify that the presence of the recombinant DNA construct and its expression of RNA to enhance the expression of the protein. The population of transgenic plants from multiple transgenic events are screened to identify the transgenic plants that exhibit enhanced yield.

### Example 11

### Use of Suppression Methods to Suppress Expression of Target Genes

This example illustrates monocot and dicot plant transformation to produce recombinant DNA constructs that are useful for stable integration into plant chromosomes in the nuclei of plant cells to provide transgenic plants having enhanced traits by suppression of the expression of target genes.

Various recombinant DNA constructs for use in suppressing the expression of a target gene in transgenic plants are constructed based on the nucleotide sequence of the gene for producing the protein that has the amino acid sequence of SEQ ID NOs: 16-17, 19, and 21, where the DNA constructs are designed to express (a) a miRNA that targets the gene for suppression, (b) an RNA that is a messenger RNA that is translated to the target protein and has a synthetic miRNA recognition site that would result in down modulation of the target protein, (c) an RNA that forms a dsRNA and which is processed into siRNAs that effect down regulation of the target protein, (d) a ssRNA that forms a ta-ssRNA which results in the production of siRNAs that effect down regulation of the target protein.

Each of the various types of recombinant DNA construct is used in transformation of a corn cell using the vector and method of Example 2 to produce multiple events of transgenic corn cell that are each regenerated into transgenic corn plants that are screened to identify that the presence of the recombinant DNA construct and its expression of RNA to suppress the expression of the protein. The population of transgenic plants from multiple transgenic events are screened to identify the transgenic plants that exhibit enhanced yield.

### Example 12

### Methods to Enhance Activity of miRNAs

This example illustrates monocot and dicot plant transformation to produce recombinant DNA constructs that are useful for stable integration into plant chromosomes in the nuclei of plant cells to provide transgenic plants having enhanced traits by enhancing the activity of miRNAs.

A recombinant DNA construct for use in enhancing the activity of a miRNA in transgenic plants are constructed based on the nucleotide sequence of a miRNA selected from SEQ ID NOs: 1, 2, and 7, where the DNA constructs are designed to express a pre-miRNA which is processed into the miRNA.

The recombinant DNA construct is used in transformation of a corn cell using the vector and method of Example 2 to produce multiple events of transgenic corn cell that are each regenerated into transgenic corn plants that are screened to identify that the presence of the recombinant DNA construct and its expression of RNA to enhance the activity of the miRNA. The population of transgenic plants from multiple transgenic events are screened to identify the transgenic plants that exhibit enhanced yield.

### Example 13

### Use of Suppression Methods to Suppress miRNA Activity

This example illustrates monocot and dicot plant transformation to produce recombinant DNA constructs that are useful for stable integration into plant chromosomes in the nuclei of plant cells to provide transgenic plants having enhanced traits by suppressing the activity of miRNAs.

A recombinant DNA construct for use in suppressing the activity of a miRNA in transgenic plants are constructed based on the nucleotide sequence of a miRNA selected from SEQ ID NOs: 9-13 and 15, where the DNA constructs are designed to express a miRNA decoy which contains a miRNA recognition site and by binding to the miRNA, sequesters it away from target genes.

The recombinant DNA construct is used in transformation of a corn cell using the vector and method of Example 2 to produce multiple events of transgenic corn cell that are each regenerated into transgenic corn plants that are screened to identify that the presence of the recombinant DNA construct and its expression of RNA to suppress the activity of the miRNA. The population of transgenic plants from multiple transgenic events are screened to identify the transgenic plants that exhibit enhanced yield.

### Example 14

### Suppression of LG1 in Corn Plants Using a Synthetic miRNA

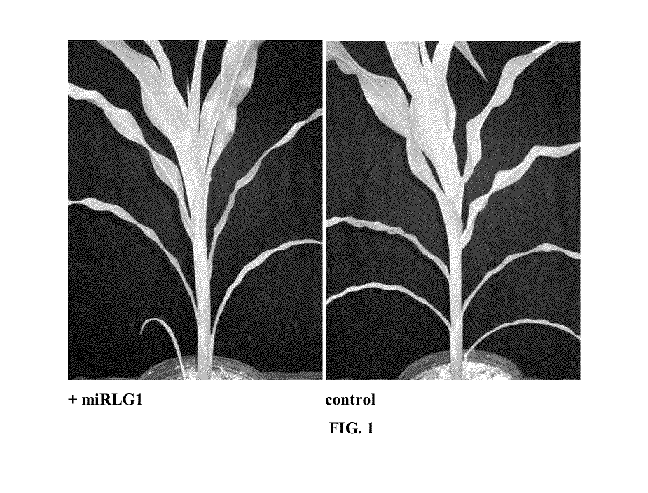

This example illustrates the use of a synthetic miRNA to suppress the expression of the native LG1 gene (SEQ ID NO: 19) in corn plants. A transgene comprising the synthetic miRNA of SEQ ID NO: 1132 was designed to suppress the expression of LG1 in transgenic corn plants. Suppression of LG1 in the plants produced plants with an altered (more vertical) leaf architecture relative to control plants lacking the transgene (FIG. 1).

1-18. (canceled)

19. A recombinant DNA construct comprising a heterologous promoter operably linked to a polynucleotide sequence that encodes a small RNA or a decoy thereof, wherein said small RNA has at least 95% identity to a sequence selected from the group consisting of SEQ ID NOs: 1-2, 7, 9-13, 15, and 895-1126.

20. The recombinant DNA construct of claim 19, wherein said polynucleotide sequence has at least 95% identity to a sequence selected from the group consisting of SEQ ID NOs: 1128 and 1135-1140.

21. A transgenic plant cell comprising the recombinant DNA construct of claim 19.

22. A transgenic plant or seed comprising a plurality of plant cells of claim 21.

23. The transgenic plant or seed of claim 22, wherein said transgenic plant or seed is from rice, wheat, or corn.

24. The transgenic plant of claim 22, wherein said transgenic plant comprises increased yield compared to a control plant.

25. A recombinant DNA construct comprising a promoter operably linked to a polynucleotide sequence encoding a polypeptide having at least 90% identity to a sequence selected from the group consisting of SEQ ID NOs: 16-21, 25-894, and 1127.

26. A transgenic plant cell comprising the recombinant DNA construct of claim 25.

27. The transgenic plant cell of claim 26, further comprising a DNA sequence encoding a protein that provides tolerance from exposure to an herbicide.

28. The transgenic plant cell of claim 26, wherein said herbicide comprises glyphosate, dicamba, or glufosinate.

29. A transgenic plant or seed comprising a plurality of plant cells of claim 26.

30. The transgenic plant or seed of claim 29, wherein said transgenic plant or seed is homozygous for said recombinant DNA construct.

31. The transgenic plant or seed of claim 29, wherein said transgenic plant or seed is from rice, wheat, or corn.

32. The transgenic plant of claim 29, wherein said transgenic plant comprises increased yield compared to a control plant.

33. A recombinant DNA construct comprising a heterologous promoter operably linked to a polynucleotide sequence, wherein said polynucleotide sequence encodes a synthetic microRNA or a trans-acting (ta) siRNA that binds to a RNA coding for a polypeptide having at least 90% identity to a sequence selected from the group consisting of SEQ ID NO: 16-21, 25-894, and 1127 and suppresses the expression of said polypeptide.

34. A transgenic plant cell comprising the recombinant DNA construct of claim 33.

35. The transgenic plant cell of claim 34, further comprising a DNA sequence encoding a protein that provides tolerance from exposure to an herbicide.

36. The transgenic plant cell of claim 35, wherein said herbicide comprises glyphosate, dicamba, or glufosinate.

37. A transgenic seed or plant comprising a plurality of plant cells of claim 34.

38. The transgenic seed or plant of claim 37, wherein said transgenic seed or plant is from rice, wheat, or corn.
